(19) United States
(12) Patent Application Publication
(10) Pub. No.: US 2007/0098722 A1
(43) Pub. Date: May 3, 2007

(54) Medicament comprising inhibitors of long pentraxin ptx3

(76) Inventors: Barbara Bottazzi, MILAN (IT); Paolo Carminati, Milan (IT); Cecilia Garlanda, Milan (IT); Alberto Mantovani, Milan (IT)

Correspondence Address: NIXON & VANDERHYE, PC, 901 NORTH GLEBE ROAD, 11TH FLOOR, ARLINGTON, VA, 22203, US

(21) Appl. No.: 10/584,292

(22) PCT Filed: Dec. 21, 2004
(86) PCT No.: PCT/IT04/00714

§ 371 Date: Jun. 23, 2006

(30) Foreign Application Priority Data
Dec. 23, 2003 (IT) RM2003A000596

(51) Int. Cl. A61K 39/395; A61K 38/17

(52) U.S. Cl. 424/145.1; 514/12

(57) ABSTRACT

The use of inhibitors of long pentraxin PTX3 for the preparation of a medicament for the prevention and treatment of autoimmune diseases and of degenerative diseases of bone and cartilage is described.

The invention described herein relates to the use of inhibitors of long pentraxin PTX3 for the preparation of a medicament for the treatment of autoimmune diseases and of degenerative diseases of bone and cartilage.

### BACKGROUND OF THE INVENTION

PTX3 is a glycoprotein capable of organising itself spontaneously in a homodecameric structure held together by disulphide bridges, which is expressed in various cell types (Bottazzi, et al., J. Biol. Chem., 1997; 272: 32817-32823), particularly in mononuclear phagocytes and endothelial cells after exposure to the inflammatory cytokines Interleukin 1beta (IL-1beta) and Tumor Necrosis Factor alpha (TNF-alpha).

PTX3 consists of two structural domains, an N-terminal unrelated to any known molecule, and a C-terminal similar to the short pentraxins such as C-reactive protein (CRP). A substantial similarity has been found between human PTX3 (hPTX3) and animal PTX3s.

For an overview of the pentraxins, see H. Gewurz, et al., Current Opinion in Immunology, 1995, 7: 54-64.

Both recombinant PTX3 and PTX3 expressed by primary cells (e.g. fibroblasts, endothelial cells and innate immunity cells) are organised mainly in a decameric structure stabilised by disulphide bridges. The single monomer of PTX3 has a molecular weight of approximately 45 kDa which can be obtained from the decameric protein through reduction of disulphide bridges and subsequent alkylation of the reduced cysteines involved in the inter-monomer interaction or through site-specific mutagenesis of the same (Bottazzi, et al., J. Biol. Chem., 1997; 272: 32817-32823).

Recent studies of patients suffering from rheumatoid arthritis have shown a significant increase in expression levels of PTX3 in sinovial fluid. This increased PTX3 expression is associated with the inflammatory processes that characterise this disease (Lucchetti, et al., Clin. Exp. Immunol. 2000; 119: 196-202).

WO 03/086380 describes the use of an inhibitor of PTX3 gene expression for the treatment of autoimmune diseases, including rheumatoid arthritis.

WO 03/086380 differs from the present invention in that it envisages the use both of completely different compounds and of a completely different inhibition method compared to the compounds and inhibition method described in the present invention.

In fact, in the present patent application PTX3 antagonists are described that are capable of directly inhibiting the biological activity of the protein (PTX3).

The person skilled in the art is familiar with the fact that the regulation (in a selective manner) of gene expression by small molecules, such as not to modify the expression of other genes involved in the inflammation, as outlined in WO 03/086380, may be difficult. Furthermore, inhibiting, at gene level, the expression of a protein that plays a fundamental role in important biological functions might give rise to unwanted effects such as, for example, an increase in susceptibility to infections and reproductive sterility.

In the medical field, there therefore remains a strongly perceived need for the availability of additional inhibitors capable of functioning as PTX3 antagonists, which are useful for the treatment of diseases according to the present invention.

### DESCRIPTION OF THE INVENTION

It has now been found that inhibitors or antagonists of PTX3 can be used to prevent and treat autoimmune diseases' and degenerative diseases of bone and cartilage.

A non-limiting example, of PTX3 inhibitors according to the present invention are monoclonal or polyclonal anti-PXT3 antibodies, while a non-limiting example of PTX3 antagonists according to the present invention are monomeric PTX3 or its peptide or peptidomimetic derivatives.

The object of the present invention is therefore the use of inhibitors or antagonists of long pentraxin PTX3, which are capable of impeding the biological activity of long pentraxin PTX3, as agents useful for the preparation of a medicament for the treatment of autoimmune diseases selected from the group consisting of: systemic lupus erythematosus (SLE), multiple sclerosis (MS), arthritis, diabetes, thyroiditis, haemolytic anaemia, atrophic orchitis, Goodpasture's disease, autoimmune retinopathy, autoimmune thrombocytopenia, myasthenia gravis, primary biliary cirrhosis, chronic aggressive hepatitis, ulcerative colitis, dermatitis, chronic glomerulonephritis, Sjögren's syndrome, Reiter's syndrome, myositis, systemic sclerosis and polyarthritis; and of degenerative bone and cartilage diseases selected from the group consisting of: osteoarthritis; osteoarthrosis; degenerative diseases of the joints; collagen deficiencies; cartilage or bone diseases characterised by endochondrial ossifications: primary arthritis, including, for example, rheumatoid arthritis, juvenile arthritis, undifferentiated chronic arthritis, and polyarthritis; secondary arthritis of autoimmune origin, including, for example, systemic lupus erythematosus arthritis, psoriasic arthritis, Crohn's disease arthritis; arthritis of dysmetabolic origin, including, for example, monosodium urate arthropathy, pyrophosphate arthropathy, calcium oxalate arthropathy; infectious arthritis, arthritis due to osteoporosis, aseptic osteonecrosis, benign and malignant bone tumours.

### DETAILED DESCRIPTION OF THE INVENTION

What is meant by “inhibitors of long pentraxin PTX3” is any monoclonal or polyclonal antibody, irrespective of its natural (human or animal), recombinant or synthetic origin, which is capable of binding PTX3 and impeding its biological activity.

An example of the preparation of monoclonal antibodies according to the present invention is described by Godine, J. W., 1986, in Monoclonal Antibodies: Principle and Practice. Academic Press, San Diego, whereas an example of the preparation of polyclonal antibodies according to the present invention is described by Harlow E. and Lane D., in Antibodies: A Laboratory Manual. Cold Spring Harbor Laboratory, 1988; Cold Spring Harbor, N.Y.

What is meant by “monomeric pentraxin” is any monomeric pentraxin, irrespective of its natural (human or animal), recombinant or synthetic origin.

What is meant by “derivative of monomeric pentraxin” is either a functional analogue of said monomeric pentraxin bearing at least one mutation and conserving the functional capability of selectively inhibiting PTX3 activity, or a peptide or peptidomimetic analogue capable of simulating linear or conformational domains of PTX3 and conserving the functional capability of selectively inhibiting PTX3 activity.

The preferred type of monomeric pentraxin is human monomeric pentraxin, the sequence of which is described in WO99/32516.

The autoimmune diseases related to abnormal activation of PTX3 are comprised in the group consisting of: systemic lupus erythematosus (SLE), multiple sclerosis (MS), arthritis, diabetes, thyroiditis, haemolytic anaemia, atrophic orchitis, Goodpasture's disease, autoimmune retinopathy, autoimmune thrombocytopenia, myasthenia gravis, primary biliary cirrhosis, chronic aggressive hepatitis, ulcerative colitis, dermatitis, chronic glomerulonephritis, Sjögren's syndrome, Reiter's syndrome, myositis, systemic sclerosis and polyarthritis.

The degenerative bone and cartilage diseases related to abnormal activation of PTX3 are comprised in the group consisting of: osteoarthritis; osteoarthrosis; degenerative diseases of the joints; collagen deficiencies; cartilage or bone diseases characterised by endochondrial ossifications: primary arthritis, including, for example, rheumatoid arthritis, juvenile arthritis, undifferentiated chronic arthritis, and polyarthritis; secondary arthritis of autoimmune origin, including, for example, systemic lupus erythematosus arthritis, psoriasic arthritis, Crohn's disease arthritis; arthritis of dysmetabolic origin, including, for example, monosodium urate arthropathy, pyrophosphate arthropathy, calcium oxalate arthropathy; infectious arthritis, arthritis due to osteoporosis, aseptic osteonecrosis, benign and malignant bone tumours.

The following example further illustrates the invention.

### EXAMPLE 1

PTX3-deficient mice were used in a murine model of collagen-induced arthritis (CIA) (Campbell, et al., Eur. J. Immunol, 2000; 30: 1568-75). The aim of the experiment was to evaluate the susceptibility of PTX3−/− mice to the induction of an arthritic phenotype.

129 sv×C57 BL/6 PTX3−/− mice were treated with 100 μg of chicken type II collagen (SIGMA) in complete Freund's adjuvant added with 250 μg of heat-inactivated M. tuberculosis in a total volume of 100 μl by multiple intradermal injections in the region proximal to the tail.

The same treatment was repeated after 21 days.

At the end of the administration period, the incidence and severity of arthritis were evaluated using an arbitrary scoring system that took account of the presence of inflamed joints and their size. The results obtained are presented in Table 1.

The greater incidence of the disease in the PTX3+/+ mice, reported in Table 1, provides evidence that the PTX3−/− mice are less susceptible to the development of collagen-induced arthritis. This finding is confirmed by the clinical score which shows a greater severity of arthritis in the PTX3+/+ mice than in the PTX3−/− mice.

The results obtained indicate that the absence of PTX3 or its inhibition is useful for the prevention and treatment of inflammatory and/or degenerative diseases of bone and cartilage.

TABLE 1
Collagen-induced arthritis in PTX3+/+ and PTX3−/− mice

| Animals | Incidence* | Clinical score^{o} |
|---|---|---|
| PTX3 +/+ | 3/5 | 10 |
| PTX3 −/− | 3/7 | 3.6 |

*Incidence at the end of the experiment (60 days after the first immunisation).
^{o}Mean clinical score of animals with arthritis at the end of the experiment.
Legend:
After the second immunisation, the presence of clinical signs of arthritis of the limbs was evaluated twice a week. Each limb involved was scored from 1 to 4; each animal could therefore obtain a maximum score of 16.

As regards the aspects relating to industrial applicability, monomeric pentraxin PTX3 or its peptide or peptidomimetic derivatives or the anti-pentraxin PTX3 antibody will be in the form of a pharmaceutical composition in which the active ingredients are solubilised and/or vehicled by pharmaceutically acceptable excipents and/or diluents, such as sterile water, carboxymethyl-cellulose or other excipients known to the expert in the sector.

Examples of pharmaceutical compositions usable for the monomeric pentraxin are the same as those described for long pentraxin PTX3 in WO 99/32516.

The compounds according to the present invention can be administered by the enteral or parenteral routes, particularly preferred pharmaceutical forms being the slow-release implant or intra-articular injection forms.

The daily dose will depend, according to the primary care physician's judgement, on the patient's weight, age and general condition.

It should be noted that the preparation of said pharmaceutical compositions, including the slow-release forms, can be done using routine techniques and instruments well known to pharmacists and to experts in pharmaceutical technology.

1. Use of inhibitors or antagonists of long pentraxin PTX3 for the preparation of a medicament for the prevention and treatment of degenerative diseases of bone and cartilage.

2. Use according to claim 1, in which what is meant by inhibitor of long pentraxin PTX3 is any monoclonal or polyclonal antibody capable of binding PTX3.

3. Use according to claim 2, in which the antibody is of natural (human or animal), recombinant or synthetic origin.

4. Use according to claim 1, in which the antagonist is monomeric PTX3, or one of its peptide or peptidomimetic derivatives that conserves the functional capability of selectively inhibiting PTX3 activity.

5. Use according to claim 4, in which the antagonist is of natural (human or animal), recombinant or synthetic origin.

6. Use according to claim 4, in which the monomeric pentraxin is of human origin.

7. Use according to claim 1, in which the autoimmune disease is selected from the group consisting of: systemic lupus erythematosus (SLE), multiple sclerosis (MS), arthritis, diabetes, thyroiditis, haemolytic anaemia, atrophic orchitis, Goodpasture's disease, autoimmune retinopathy, autoimmune thrombocytopenia, myasthenia gravis, primary biliary cirrhosis, chronic aggressive hepatitis, ulcerative colitis, dermatitis, chronic glomerulonephritis, Sjögren's syndrome, Reiter's syndrome, myositis, systemic sclerosis and polyarthritis.

8. Use according to claim 1, in which said degenerative bone or cartilage disease is selected from the group consisting of: osteoarthritis; osteoarthrosis; degenerative diseases of the joints; collagen deficiencies; cartilage or bone diseases characterised by endochondrial ossifications: primary arthritis, including, for example, rheumatoid arthritis, juvenile arthritis, undifferentiated chronic arthritis, and polyarthritis; secondary arthritis of autoimmune origin, including, for example, systemic lupus erythematosus arthritis, psoriasic arthritis, Crohn's disease arthritis; arthritis of dysmetabolic origin, including, for example, monosodium urate arthropathy, pyrophosphate arthropathy, calcium oxalate arthropathy; infectious arthritis, arthritis due to osteoporosis, aseptic osteonecrosis, benign and malignant bone tumours.
